(19) United States
(12) Patent Application Publication
(10) Pub. No.: US 2002/0045912 A1
(43) Pub. Date: Apr. 18, 2002

(54) Fluid jet blood sampling device and methods

(76) Inventor: Keith D. Ignotz, Duluth, GA (US)

Correspondence Address: THORPE NORTH WESTERN, 8180 SOUTH 700 EAST, SUITE 200, P.O. BOX 1219, SANDY, UT, 84070, US

(21) Appl. No.: 09/916,123

(22) Filed: Jul. 25, 2001

Related U.S. Application Data
(63) Continuation of application No. 09/495,937, filed on Feb. 2, 2000, now abandoned.
Application No. 09/495,937 is a continuation-in-part of application No. 08/869,214, filed on Jun. 4, 1997.

(51) Int. Cl. A61B 17/32

(52) U.S. Cl. 606/167

(57) ABSTRACT

A fluid jet blood sampling system and method for obtaining a blood sample from a patient, utilizes a short duration fluid jet from a disposable jet nozzle to perforate at least the epidermis of the skin and to break at least one blood vessel, thereby allowing a quantity of blood to accumulate on the skin surface. A cowling prevents contamination of the local environment with excess fluid, blood, etc. A drying unit may be provided to remove excess fluid, blood, etc. from the skin within the cowling. A sample of blood may be transferred to a test strip, or a collection unit may be used to collect and contain a blood sample from the mammal.

This application is a continuation-in-part of U.S. patent application Ser. No. 08/869,214, which was filed Jun. 4, 1997, the contents of which are hereby incorporated by reference.

### BACKGROUND OF THE INVENTION

1. Field of the Invention

This invention relates to a needle-less apparatus and method for blood sampling from a patient, and in particular, to a fluid jet blood sampling system and method for obtaining a blood sample.

2. Background of the Related Art

Biological and biochemical analysis and testing of patients' blood is routinely performed to provide a vast array of diagnostic information concerning a patient's state of health. Consequently, blood sampling is an everyday occurrence in literally millions of medical establishments world-wide. In addition, blood sampling is also a commonly performed procedure in veterinary medicine and biomedical research.

Conventional blood sampling devices and methods involve perforating the skin of a patient with a lancet, a needle, or other sharp mechanical instrument (sharps). Needles, lancets, and the like will easily penetrate human skin as a result of accidental or casual contact with such sharps. In recent years there has been increasing concern over the risk to medical professionals of contracting serious blood-borne diseases (e.g. AIDS) by being accidentally cut or poked by sharps bearing contaminated (infectious) blood. There is also concern over similar risks to janitorial/disposal personnel who may be exposed to contaminated sharps in laboratory or hospital waste, and to members of the public who may be exposed to contaminated sharps which may have been improperly disposed of.

A number of attempts have been made to provide a blood sampling apparatus and/or method which produces perforation of the skin without the use of sharps. For example, U.S. Pat. No. 5,165,418 to Tankovich and U.S. Pat. No. 5,554,153 to Costello both disclose the use of laser devices for perforating the skin to permit blood samples to be drawn. Such devices, comprising a laser, are relatively bulky, immobile, and expensive.

Fluid jet technology has been used for various surgical procedures, such as for cutting tissue, penetrating the skin, and injecting substances into the body of a patient. For example, U.S. Pat. No. 4,560,373 discloses a surgical nozzle apparatus for performing operations such as dissection, resection, and cutting by means of a continuous jet of fluid. The apparatus includes a handpiece incorporating a nozzle piece for injecting a stream of a fluid under controlled pressure from a source of pressurized fluid, a valve assembly adapted to cut off the supply of the pressurized fluid to the nozzle piece, and a suction nozzle including a suction pipe connected to external suction means.

U.S. Pat. No. 5,505,697 discloses an improved fluid jet injection method and device for piercing the skin and for injecting a substance into a patient. The device includes a plunger driver which impacts against a plunger to generate a high initial pressure pulse for piercing the skin. A lower delivery phase pressure is used during injection of the substance.

U.S. Pat. No. 5,505,729 discloses a process and arrangement for high pressure fluid jet selective cutting of tissue, in which improved cutting speed is achieved, concomitant with minimal bleeding of the tissue by use of a high frequency electro-coagulator in combination with prior art liquid jet surgical cutting.

U.S. Pat. No. 5,599,302 discloses a needle-less injection system which includes a portable, hand-held device for injecting a medical product into a patient, the device having a nozzle assembly. The injection system of the '302 patent includes an energy device for pushing the medical product out of the nozzle assembly, and an energy device actuating mechanism. The energy device is a self-contained gas spring operatively connected to a plunger.

U.S. Pat. No. 5,562,692 discloses a pulsed fluid jet surgical tissue cutting/emulsifying and aspirating tool, in which a pressure intensifier piston arrangement functions in conjunction with a relatively low pressure fluid supply, a relatively low pressure gas supply, and a relatively high pressure relief valve, to pump a fluid jet as a series of high pressure pulses.

The possible use of a hydraulic jet or a high pressure jet of fluid has been disclosed in the context of methods to increase the permeability of the skin. For example, U.S. Pat. No. 5,445,611 to Eppstein et al. discloses methods for enhancing the permeability of the skin, with particular reference to the outermost layer of the skin (stratum corneum), to a permeant, drug, or pigment by the use of ultrasound, for the purpose of promoting uptake of substances into the skin or through the skin. The '611 patent mentions the use of a hydraulic jet as one of several possible mechanisms for perforating the skin in order to augment ultrasound induced increased skin permeability. However, the '611 patent does not teach the removal of a sample of blood, nor the removal of any other bodily fluid, molecules, cells, or tissue, from the body.

U.S. Pat. No. 5,458,140 also to Eppstein et al., discloses methods for enhancing the permeability of the skin or mucosa (mucous membranes) to an analyte by the use of ultrasound, with or without the presence of a chemical enhancer, for the purposes of monitoring the analyte. The '140 patent mentions the use of a hydraulic jet as one of several possible mechanisms to form perforations in the stratum corneum in order to augment ultrasound induced enhanced skin permeability. However, the '140 patent does not teach the use of a fluid jet device as a means for forming a hole in the skin of an individual such as to permit the drawing of blood from that individual. Further, the '140 patent does not teach a method for obtaining blood from an individual. Nor does the '140 patent disclose an apparatus or method for forming a hole in the skin of an individual of sufficient width and/or depth to provide a quantity of blood; nor does the '140 patent disclose the collection of a blood sample from an individual, by means of a fluid jet, or otherwise.

The contents of U.S. Pat. Nos. 4,560,373; 5,165,418; 5,505,697; 5,505,729; 5,554,153; 5,599,302; 5,562,692; 5,445,611; and 5,458,140 are hereby incorporated by reference in their entirety for their teachings of additional or alternative details, features and/or technical background.

### SUMMARY OF THE INVENTION

The instant invention provides a needle-less fluid jet device for penetrating the skin to permit a blood sample to be drawn. A device embodying the invention is relatively inexpensive to purchase and maintain, is compact, can be easily and quickly operated by an unskilled medical professional with minimal pain to the patient. A device embodying the invention may include disposable components which can be easily replaced and are safe to handle by medical and non-medical personnel. Such a blood sampling device has several advantages over conventional blood sampling means (lancet or needle), and also greatly reduces or eliminates the risk of persons being accidentally infected with a blood-borne pathogen from a contaminated sharp instrument.

An object, therefore, of the invention is to provide a blood sampling system which emits one or more short duration fluid jets to penetrate the skin to allow a sample of blood to be drawn. The pressure and/or duration of the fluid jets may be adjustable. The fluid jet may penetrate the skin to allow a drop of whole blood to accumulate on the skin surface. The fluid jet may break a blood vessel beneath the surface of the skin to allow the blood to accumulate on the skin surface.

Blood that collects on the skin surface could be easily transferred to a collection vessel or a collection strip. Alternatively, a device embodying the invention may include a collection unit for collecting and retaining a blood sample, and the collection unit may comprise one or more capillary tubes arranged within a cowling.

Another object of the invention is to provide a blood sampling system which includes a disposable, integrated jet nozzle/cowling. The jet nozzle/cowling may be formed of a single piece of molded plastic. The jet nozzle/cowling may be disposable. The jet nozzle/cowling may be configured to surround the patient's skin at the locus where blood is to be drawn to prevent the release of blood, as droplets or as an aerosol, from the blood sampling locus.

At least one capillary tube may be provided within or adjacent to the cowling to remove excess jet fluid, first drawn or diluted blood, and other unwanted material from the skin surface. The device may also include a drying unit for removing unwanted liquid from a locus on the skin whence blood is to be drawn. The jet nozzle/cowling may further include a valve to prevent the contamination of upstream components of the blood sampling system with blood from a patient.

A device embodying the invention may use a battery or spring operated piston to generate pressurized fluid for the fluid jet. In other embodiments, the device may include a plunger for generating the fluid jet, where the plunger is adjustable to vary the jet pressure. In still other embodiments, the device may utilize a disposable source of compressed gas or fluid for generating the fluid jet, like a CO₂ cartridge.

A method embodying the invention, for obtaining a blood sample from beneath a surface of skin, may include the steps of: propelling a short duration fluid jet at a locus on the skin to form a hole in the skin; allowing at least one drop of blood to accumulate on the skin surface; and collecting a sample of the at least one drop of blood. In a method embodying the invention, the short duration fluid jet may operate to break at least one blood vessel beneath the perforated skin at the locus, thereby allowing a first quantity of blood to accumulate at the locus. The method may include a step of adjusting the pressure, duration, or volume of fluid of the fluid jet.

A method embodying the invention may also include the steps of allowing a first quantity of blood and fluid to accumulate at the locus; removing the first quantity of blood and fluid from the locus; allowing the accumulation of a second quantity of blood at the locus; and collecting a sample of the second quantity of blood. The first quantity of blood and fluid may be removed by a drying unit.

In methods embodying the invention, the blood sample may be collected by transferring a sample of the quantity of blood to a test strip. Alternatively, the blood sample may be collected in a collection vessel, such as a capillary tube. The collection vessel may be a removable portion of a device used to propel the short duration fluid jet, or the collection vessel may be completely separate from the fluid jet device.

Additional advantages, objects, and features of the invention will be set forth, in part, in the description which follows, and, in part, will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawing figures, wherein like reference numerals refer to like elements, and wherein:

FIG. 1A is a block diagram that illustrate a fluid jet blood sampling system according to one embodiment of the invention;

FIG. 1B is a block diagram that illustrates a fluid jet blood sampling system according to another embodiment of the invention;

FIG. 1C is a block diagram that illustrate a fluid jet blood sampling system according to another embodiment of the invention;

FIG. 2A shows a partial perspective view of a disposable, integrated fluid jet nozzle/cowling for use in conjunction with a fluid jet blood sampling system embodying the invention;

FIG. 2B is a cross-sectional view of the disposable, integrated fluid jet nozzle/cowling of FIG. 2A;

FIG. 2C is a view of the disposable, integrated fluid jet nozzle/cowling as seen from line 2C-2C of FIG. 2A;

FIG. 2D shows a schematic representation of a fluid jet blood sampling system according to another embodiment of the invention;

FIG. 2E shows a view of another embodiment of the disposable, integrated fluid jet/cowling as seen from line 2C-2C of FIG. 2A;

FIG. 3A shows a schematic representation of a fluid jet blood sampling system according to another embodiment of the invention;

FIG. 3B is a schematic representation of a fluid jet blood sampling system according to another embodiment of the invention, in which a drying duct is coupled to a trap unit for trapping unwanted liquid(s);

FIG. 3C shows a fluid jet blood sampling system according to another embodiment of the invention in which the fluid jet blood sampling system includes a separation unit for separating or stripping fluid of the fluid jet from the cellular fraction of blood;

FIG. 3D shows a fluid jet blood sampling system according to another embodiment of the invention in which a single vacuum unit is used both for removing unwanted liquid and for collecting a blood sample;

FIG. 4A is a schematic representation of a fluid jet unit of a fluid jet blood sampling system according to another embodiment of the invention;

FIG. 4B is a schematic representation of a cowling or hood of a fluid jet blood sampling system according to another embodiment of the invention;

FIG. 5A is a frontal view of a fluid jet unit according to another embodiment of the invention, showing a jet nozzle and a cowling attached to a barrel portion of the fluid jet unit, the cowling having a pair of collection coupling pieces;

FIG. 5B shows a frontal view of a fluid jet unit, as shown in FIG. 5A, but in which the cowling has a single collection coupling piece together with a single drying coupling piece;

FIGS. 6A-6F schematically represent steps involved in methods for assembling a fluid jet blood sampling system, according to various embodiments of the invention;

FIGS. 7A and 7B schematically represent steps involved in methods for collecting a blood sample using a fluid jet blood sampling system, according to embodiments of the invention;

FIG. 8 schematically represents steps involved in a method for obtaining a blood sample using a fluid jet blood sampling system, according to another embodiment of the invention;

FIG. 9 schematically represents steps involved in a method for obtaining a blood sample using a fluid jet blood sampling system, according to another embodiment of the invention;

FIG. 10 schematically represents steps involved in a method for obtaining a blood sample from a patient, using a fluid jet blood sampling system, according to another embodiment of the invention; and,

FIG. 11 schematically represents steps involved in a method for assembling a fluid jet blood sampling system, according to another embodiment of the invention.

### DETAILED DESCRIPTION OF PREFERRED EMBODIMENTS

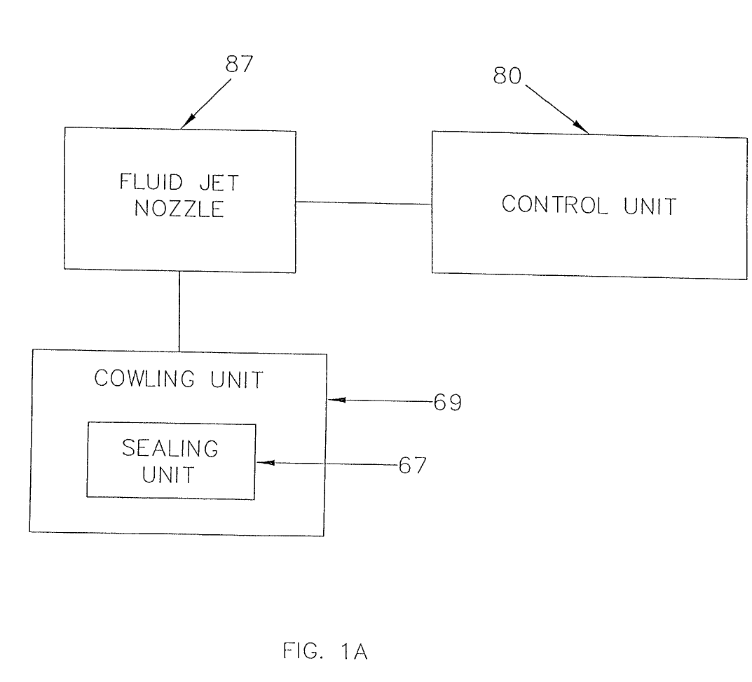

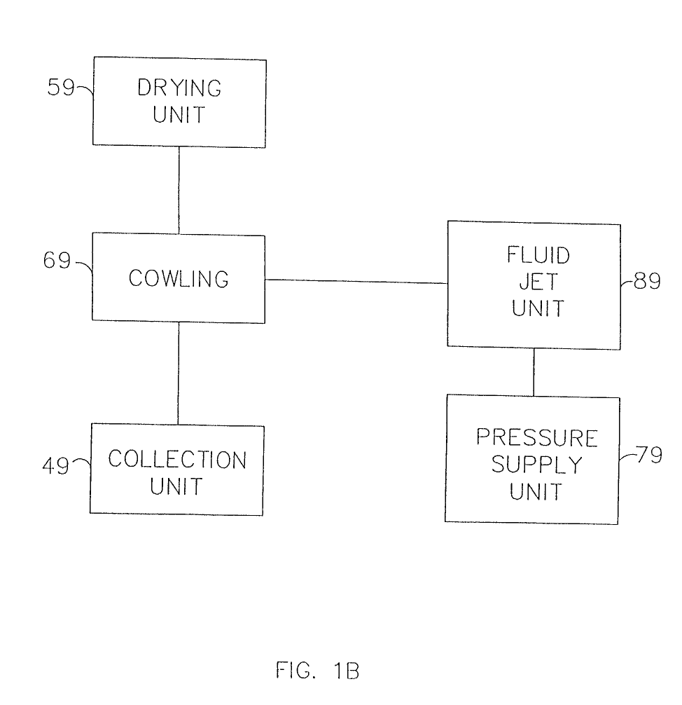

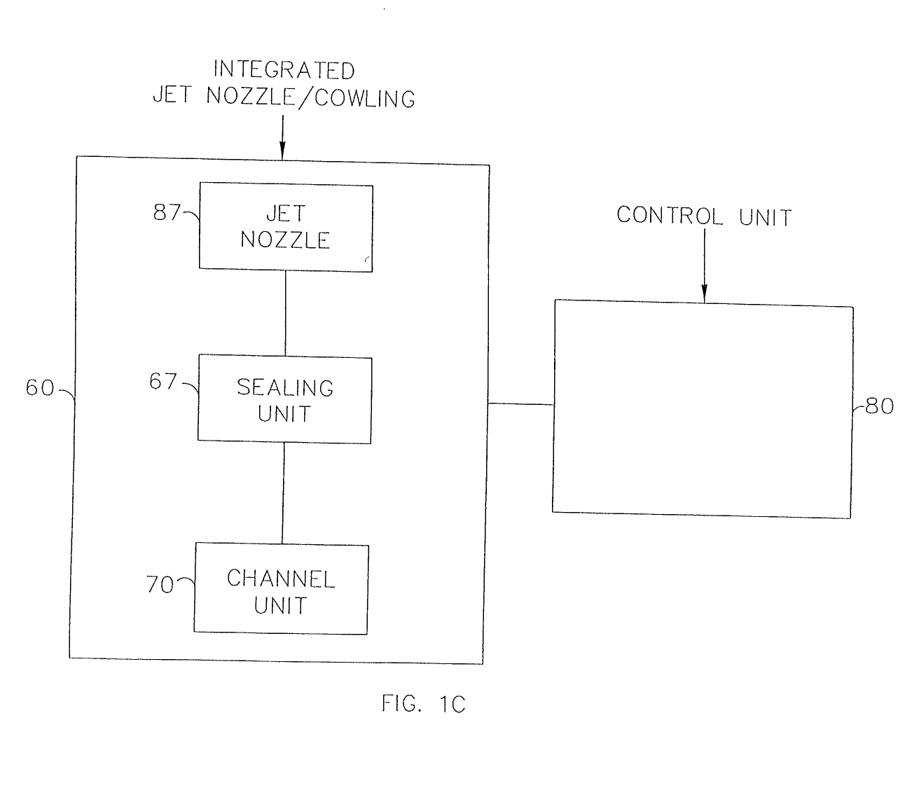

FIG. 1A is a block diagram that schematically represents a fluid jet blood sampling system according to one embodiment of the invention. The fluid jet blood sampling system includes a control unit 80, which allows adjustment of the quantity, pressure or duration of the jet of fluid released from a fluid jet nozzle 87. The fluid jet nozzle 87 delivers one or more a short duration fluid jets for penetrating skin and/or breaking a blood vessel beneath the skin. This allows at least one drop of blood to accumulate on the surface of the skin.

The duration of each of the fluid jets may be only a fraction of a second. However, several fluid jets may be emitted over a period of several seconds during a blood sampling operation. The quantity of fluid in each fluid jet can be extremely small, being measured in milliliters or fractions of a milliliter. The duration of each fluid jet, the volume of fluid in each fluid jet and the pressure at which the fluid jets are emitted can be varied without departing from the invention, provided the fluid jets are capable of penetrating the patient's skin and breaking a blood vessel so that blood can accumulate on the skin surface. In some embodiments of the invention, the device may be adjustable so that a user can vary the pressure at which fluid jets are emitted, the number of fluid jets that are emitted during a single blood sampling procedure, and/or the volume of fluid in each fluid jet. This would allow a user to adjust the device to provide the minimum amount of force necessary to effectively bring blood to the skin surface considering the condition of the skin of the patient.

The system also includes a cowling unit 69 having a sealing unit 67 for sealing the area of contact between cowling unit 69 and the skin surface. As will be described more fully hereinbelow, according to one embodiment of the invention, jet nozzle 87 and cowling unit 69 may be integrated and constructed as a single component. The cowling unit 69 having sealing unit 67 is for preventing splashing or distribution of fluid from the fluid jet, blood, etc. from the skin surface.

After some blood has accumulated on the surface of the skin, the blood may be collected or transferred to a secondary device, such as a test strip, collection vessel, or similar device (not shown). The collection device may include, for example, a bottle, tube, or other container made of glass or various types of plastic materials, etc. including a tube having a relatively narrow bore, such as a glass capillary tube, or the like. The collected blood can then be tested.

According to still another embodiment of the invention, after some blood has accumulated on the surface of the skin, the blood may be collected via a collection unit (also not shown in FIG. 1A) that is integral to the fluid jet blood sampling system. According to certain embodiments of the invention, a collection unit may, for example, take the form of at least one collection capillary for collecting and retaining a relatively small volume of blood. Herein the term “collection capillary” may be used to refer to one or more relatively small diameter (e.g., capillary) tubes or a channel having a relatively narrow width, and being suitable for receiving, collecting, and/or retaining a sample of blood from a patient. A collection capillary, or other form of collection unit, according to the invention, may be housed within the cowling or be integral with the cowling, as described hereinbelow.

Analogously, the term “drying capillary” may be used to refer to one or more relatively small diameter (e.g., capillary) tubes or a channel having a relatively narrow width, and being suitable for removing, receiving, and/or retaining unwanted material (e.g., excess fluid, first drawn blood, etc.) from the skin surface of a patient. A drying capillary, or other form of drying unit, according to the invention, may be housed within the cowling and indeed may be integral with the cowling, as described hereinbelow.

Herein the term “capillary” may be used to refer generically to one or more relatively narrow bore or small diameter (e.g., capillary) tubes, or to a channel having a relatively narrow width, and being suitable for use as a collection capillary or as a drying capillary.

FIG. 1B is a block diagram to schematically represent a fluid jet blood sampling system according to another embodiment of the invention. In this embodiment, a fluid supply unit (not shown) for supplying a fluid is connected to a pressure supply unit 79 for pressurizing the fluid. According to one embodiment of the invention, pressurized fluid for generating a fluid jet may be generated by a battery powered device. The pressure supply unit 79 may include a piston, for example, a piston which reciprocates within a cylinder having at least one valve. Such battery powered devices are well known in the art. Alternatively, the pressure supply unit 79 may utilize a force of a spring to pressurize the fluid. In still another embodiment, the pressure supply unit 79 may utilize a supply of pressurized gas, such as a disposable CO₂ cartridge, to pressurize the fluid jet.

The fluid supplied by the fluid supply unit 79 may be sterile distilled water, filtered and/or deionized water, or a sterile physiological saline, various buffered solutions or buffers, or other suitable fluids. The fluid may also include an anti-bacterial agent, an anti-infective agent, or an anesthetic.

As shown in FIG. 1B, the fluid jet unit 89 is for generating a fluid jet capable of breaching skin in order to provide a quantity of blood, and preferably to allow at least one drop of blood to accumulate on the surface of the skin. Fluid jet unit 89 may have a cowling (or hood) 69 attached thereto. A collection unit 49 may be coupled to the cowling 69. A drying unit 59 may also be coupled to the cowling 69. It should be noted that the term “drying unit” as used herein may include one or more capillary tubes (not shown in FIG. 1B) which serve to remove unwanted materials from the surface of skin and/or from within cowling 69.

FIG. 1C is a block diagram to schematically represent a fluid jet blood sampling system according to another embodiment of the invention. In this embodiment, a control unit 80 is functionally coupled to an integrated jet nozzle/cowling 60. Control unit 80 provides pressurized fluid to integrated jet nozzle/cowling 60, the latter including a jet nozzle 87, a sealing unit 67, and a channel unit 70. The pressurized fluid is provided to the integrated jet nozzle/cowling 60 at a pressure sufficient to pierce the skin of a patient. For instance, the fluid may be provided at a pressure in the range of from about 1,000 to 15,000 psi (pounds per square inch). Preferably, the pressurized fluid is provided to integrated jet nozzle/cowling 60 at a pressure in the range of from about 8,000 to 11,000 psi.

The control unit may be used to control the duration of a fluid jet, the pressure at which the fluid is provided, and the quantity of fluid within a jet. In addition, the control unit 80 may be configured to provide the fluid such that a plurality of short duration fluid jets are emitted by the jet nozzle 87.

Nozzle 87 provides a pressurized fluid jet capable of forming a hole through skin and breaking at least one blood vessel, thereby allowing at least one drop of blood to accumulate at the surface of the skin adjacent to the hole formed in the skin. Sealing unit 67 effectively forms a seal between the surface of the skin and integrated jet nozzle/cowling 60, thereby preventing release of materials, such as excess fluid from the fluid jet, first-drawn blood, etc., from within sealing unit 67 to the exterior. Channel unit 70 may serve to withdraw material from within sealing unit 67 of integrated jet nozzle/cowling 60. Materials which may be withdrawn from within sealing unit 67 of integrated jet nozzle/cowling 60 by channel unit 70 include excess fluid from the fluid jet, first-drawn blood, etc.

Materials may be withdrawn from within sealing unit 67 by channel unit 70 as a result of various forces acting alone or in combination. Forces which may be involved in withdrawal of materials from within sealing unit 67 by channel unit 70 include, but are not restricted to, back pressure from the fluid jet emanating from jet nozzle 87, capillary action, and suction, such as suction from a vacuum pump or aspirator device. Capillary action refers to the greater attraction between a liquid and a solid surface as compared with the cohesive forces within the liquid itself.

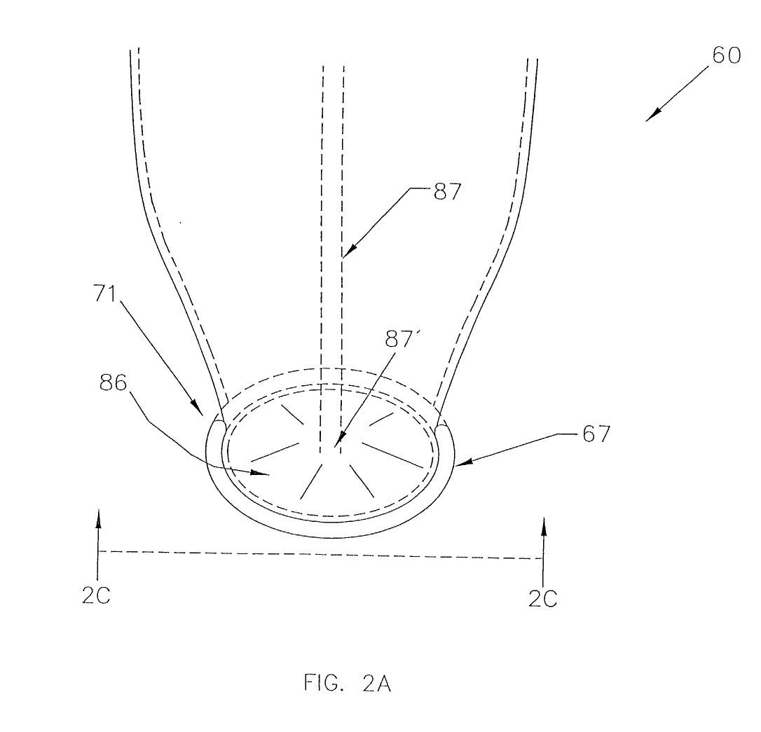

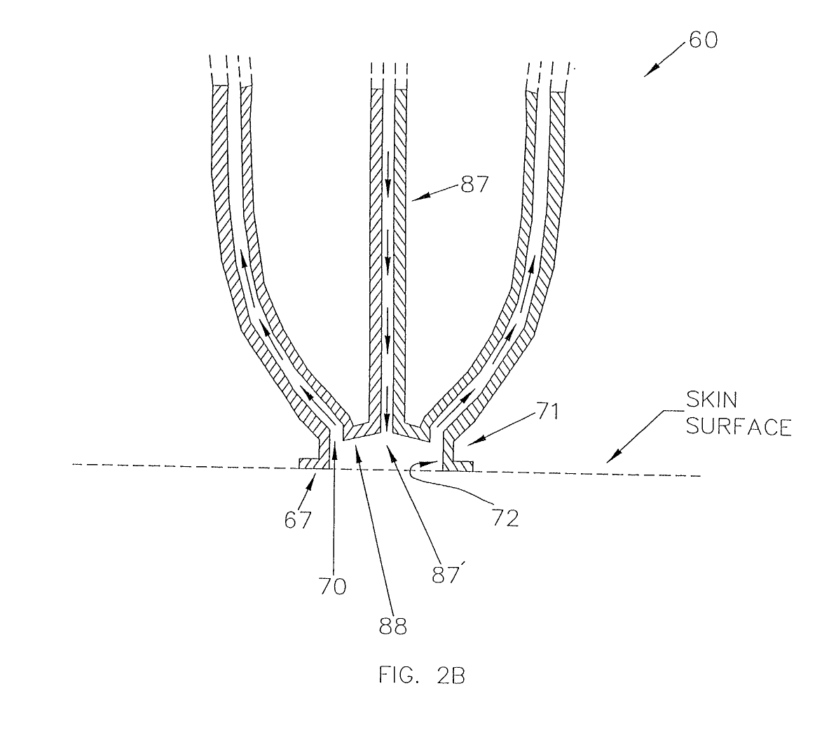

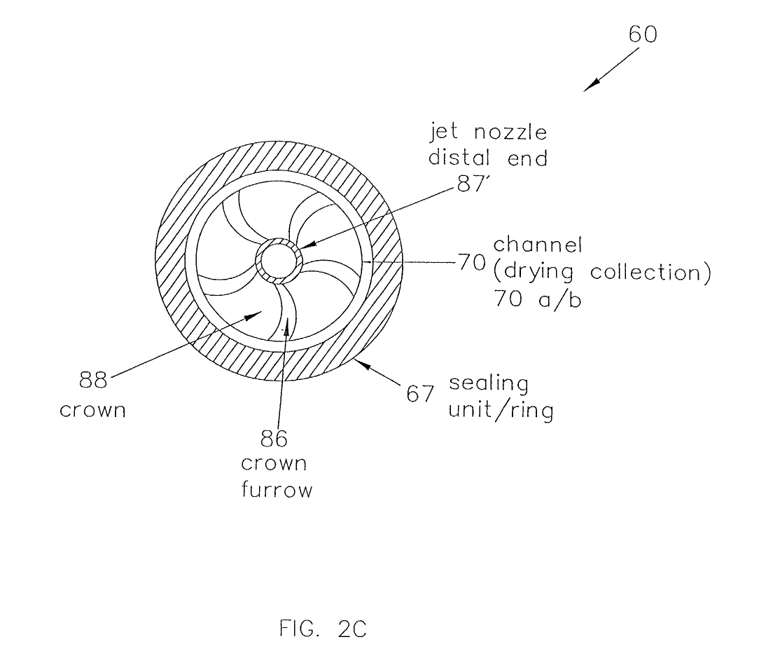

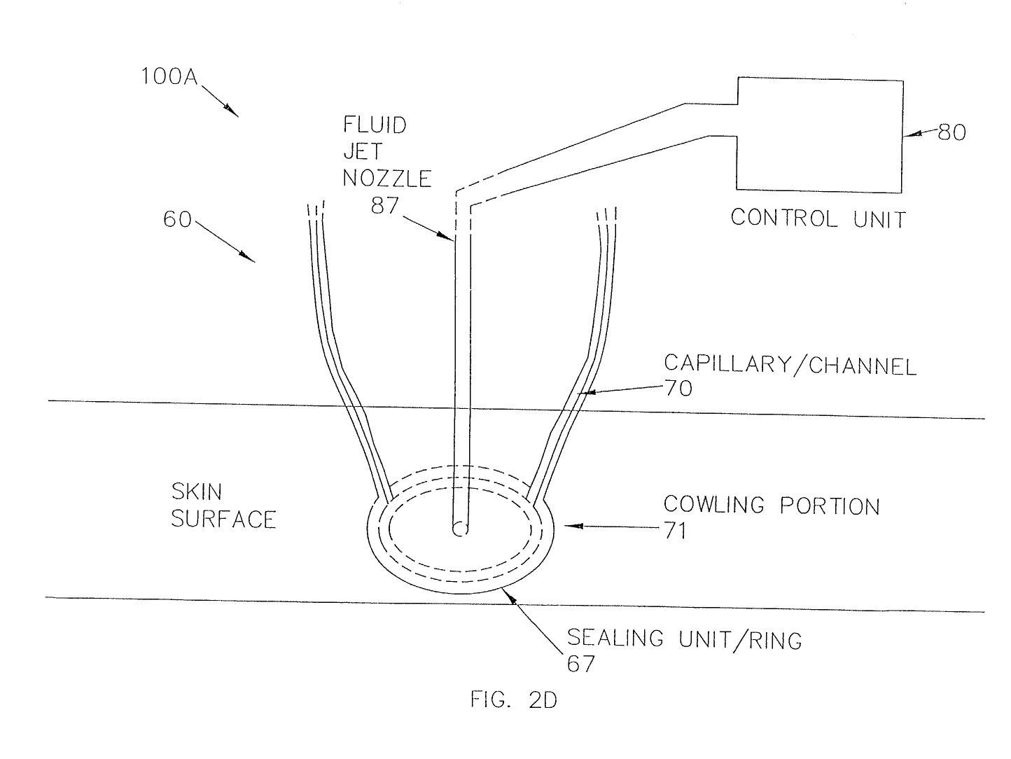

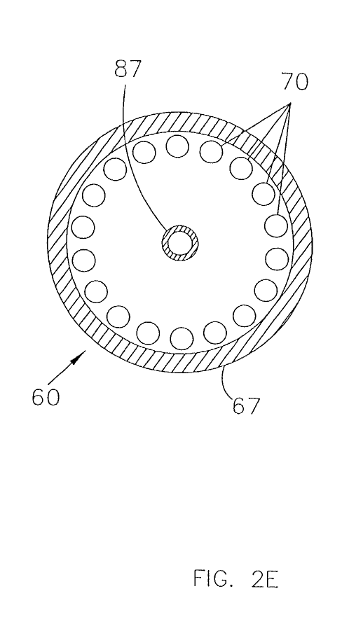

FIG. 2A shows a partial perspective view of an integrated fluid jet nozzle/cowling 60 for use in conjunction with a fluid jet blood sampling system, according to another embodiment of the invention. Integrated fluid jet nozzle/cowling 60 includes a jet nozzle 87 for providing a pressurized fluid jet. Jet nozzle 87 includes jet nozzle distal end 87′. A sealing unit or sealing ring 67 forms a seal against the skin surface with which integrated fluid jet nozzle/cowling 60 is brought in contact, thereby preventing escape of materials from within integrated fluid jet nozzle/cowling 60.

A channel or channel unit 70 (not shown in FIG. 2A) may be provided to remove unwanted materials, such as excess fluid jet fluid, first-drawn blood, or a mixture of these, etc., which might otherwise accumulate within integrated fluid jet nozzle/cowling 60. Integrated fluid jet nozzle/cowling 60 may further include a crown 86, which may take the form of a domed ceiling. The crown could extend in a circular fashion from the perimeter of jet nozzle distal end 87′ to channel or channel unit 70 (FIGS. 2A, 2C).

As shown in FIGS. 2A and 2B, a cowling portion 71 of integrated jet nozzle/cowling 60 may include an inner wall 72, crown 88, and sealing ring 67. Preferably, integrated fluid jet nozzle/cowling 60, including channel 70, jet nozzle 87, and sealing ring 67, is of unitary construction of a single piece of plastic material, or the like, such as may be formed by a molding process. Plastic materials which may be suitable for construction or molding of integrated fluid jet nozzle/cowling 60 include medical plastic materials such as polyethylene, polypropylene, or Medi-Jector Choice, which is made by Medi-Ject, Corp. of Minneapolis, Minn.

An integrated fluid jet nozzle/cowling 60 made from molded plastic would be constructed so as to be readily coupled/uncoupled or attached/detached from other components of a fluid jet blood sampling system. Preferably the integrated fluid jet nozzle/cowling would be attached to the control unit via a “snap-and-break” connection so that it can only be used one time. This helps to prevent the spread of infection because the integrated fluid jet/nozzle cannot be used on two separate patients.

The disposable integrated fluid jet nozzle/cowling 60 is inexpensive to produce and cost-competitive as compared with conventional lancets or sharps. Furthermore, the integrated fluid jet nozzle/cowling 60 is more conveniently disposed of after use because it is not sharp, and therefore does not present a skin rupture threat to medical or janitorial personnel.

FIG. 2B shows the relationship between the channel 70, the jet nozzle distal end 87′, the jet nozzle 87, the crown 88, and the sealing ring 67. In use, the sealing ring 67 makes contact with the skin surface, while the channel 70 and the distal end of jet nozzle 87′ are situated above the skin surface to a greater or lesser extent. The longer arrows indicate the direction of flow of pressurized fluid within the fluid jet, while the shorter arrows indicate the flow of materials within the channel 70. Channel 70 is located towards the perimeter of sealing rim 67. Channel 70 may take the form of a continuous circular channel located within integrated fluid jet nozzle/cowling 60, or may constitute one or more relatively small bore tubes or capillaries, as seen in FIG. 2E.

Jet nozzle 87 may have a diameter ranging from about 10 μm to about 500 μm. Preferably, jet nozzle 87 has a diameter ranging from about 50-100 μm, and more preferably jet nozzle 87 has a diameter of about 80 μm. Channel 70 may have a width ranging from about 50 μm to several mm in width. In situations where capillary action, or capillary attraction, are the primary forces involved in withdrawing materials from within sealing ring 67 via channel 70, the width of channel 70 is preferably in the range of from about 100 μm to about 500 μm.

FIG. 2C is a view of a disposable integrated fluid jet nozzle/cowling 60 as seen from line 2C-2C of FIG. 2A. In a preferred embodiment of the invention, the sealing ring 67 is substantially circular. The channel 70 is also substantially circular and lies within, or internal to, the sealing ring 67 and in close proximity to the inner circumference of the sealing ring 67. The jet nozzle 87 may terminate at the jet nozzle distal end 87′ in approximately a central location corresponding to approximately the center of a concentric circle formed by the channel 70 and the sealing ring 67. The channel 70 may serve as a drying channel 70a to withdraw unwanted materials, such as excess fluid jet fluid, first drawn blood, etc., from within the sealing ring 67. Thereafter, at least one drop of blood may be allowed to accumulate on the skin surface, prior to sampling or collection of the blood with a secondary device, such as a test strip or small tube. The integrated fluid jet nozzle/cowling 60 may be removed from the skin prior to collection of the blood with a secondary device. The channel 70 may also serve as a collection channel 70b to withdraw a sample of blood from within the sealing ring 67. The overall diameter or external diameter of the sealing ring 67 may range from about 0.5 to about 2.5 inches. The crown 88 may include one or more crown furrows 86 or topographical features which may serve to promote the flow of liquid materials towards the channel 70.

FIG. 2D shows a schematic representation of a fluid jet blood sampling system 100A according to another embodiment of the invention. FIG. 2D is not drawn to scale. According to FIG. 2D, the integrated jet nozzle/cowling 60 is functionally coupled to and controlled by a control unit 80, such as a time/pressure control unit. The control unit 80 may control the length or duration of a fluid pulse, the pressure of the fluid and the incident angle of the pulse. The control unit may also control the volume of fluid included in a fluid jet. Further, the control unit may cause a device embodying the invention to deliver only a single short duration fluid jet, or a series of short duration fluid jets. Where multiple fluid jets are delivered, the fluid jets can have the same or different fluid volumes, durations or pressures.

One or more capillaries or channels 70 may be provided to remove excess fluid from the fluid jet, first drawn blood (usually less than 1 ml.), and other unwanted materials from the skin surface within sealing unit or ring 67 of cowling portion 71. Herein the terms “drying capillary” and “drying capillary tube” will be used to refer to a capillary tube suitable for receiving, collecting, and/or removing unwanted material (e.g., excess fluid, skin fragments, first drawn blood, etc.) from the skin surface of a patient. One or more additional capillaries 70 (collection capillaries) may also be provided for collecting a portion of the at least one drop of blood accumulated at the surface of the skin. Both collection capillaries and drying capillaries, when provided, may be conveniently arranged at one or more suitable locations within integrated jet nozzle/cowling 60. FIG. 2E shows an integrated jet nozzle/cowling 60 that includes a plurality of capillary tubes 70 arranged around a central fluid jet nozzle 87.

According to an alternative embodiment of the invention, blood accumulated at the surface of the skin may be transferred to one or more test strips (not shown). Test strips for various qualitative and quantitative biochemical and physiological analyses are well known in the art. For instance, the Chemstrip bG, manufactured by Boeringer Mannheim of Indianapolis, Ind., is used to test a blood glucose level.

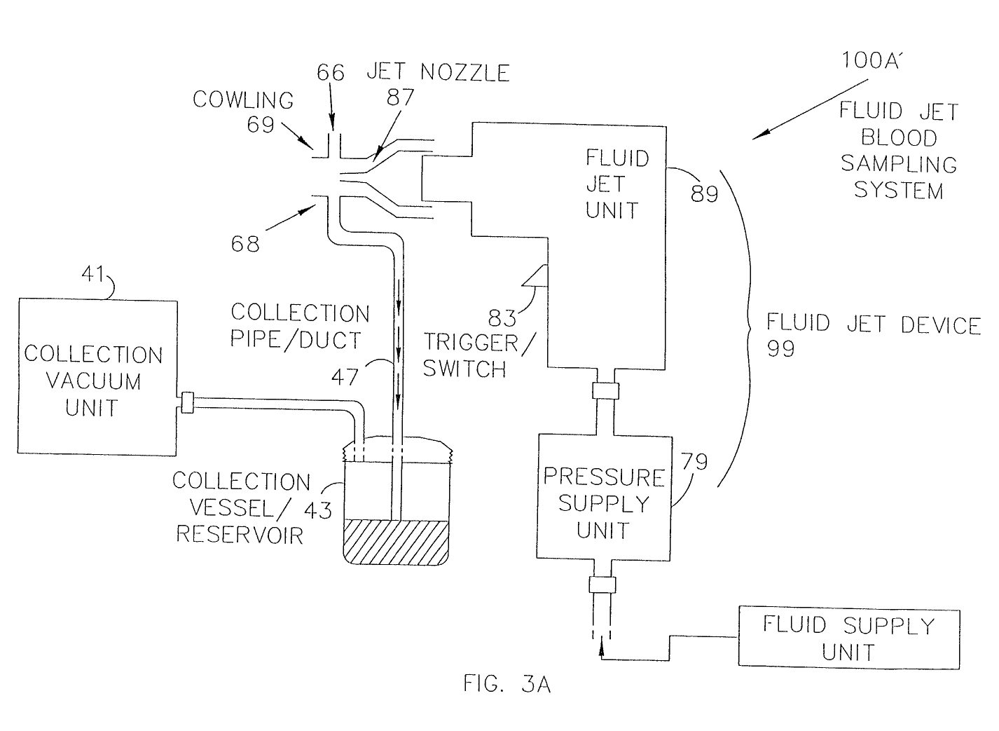

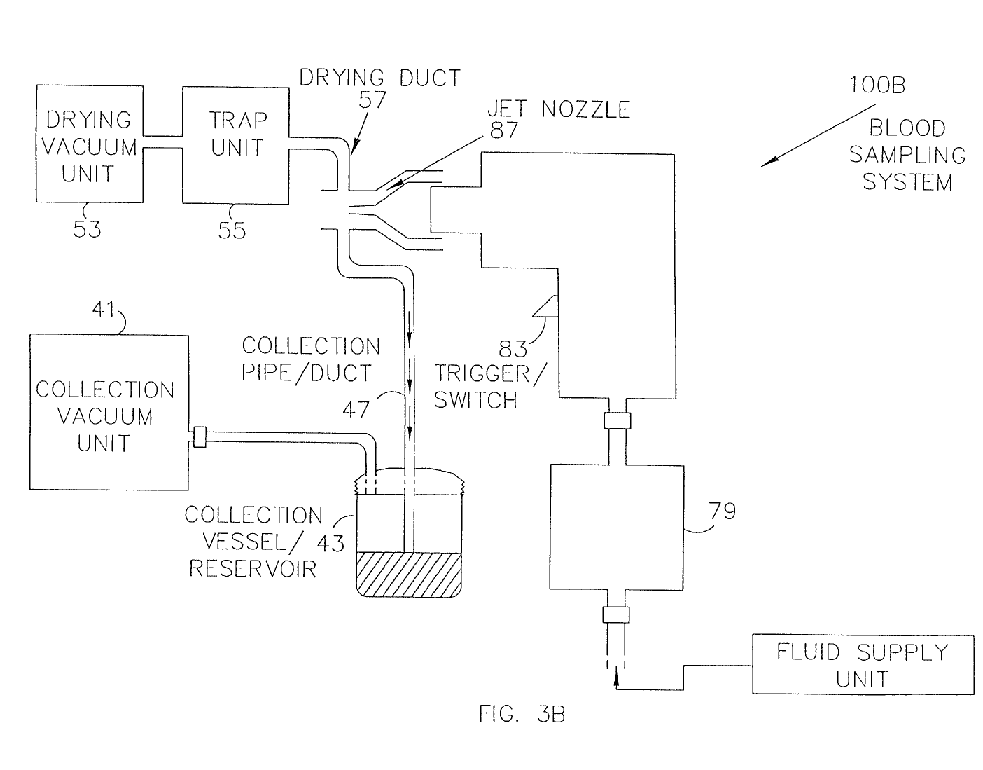

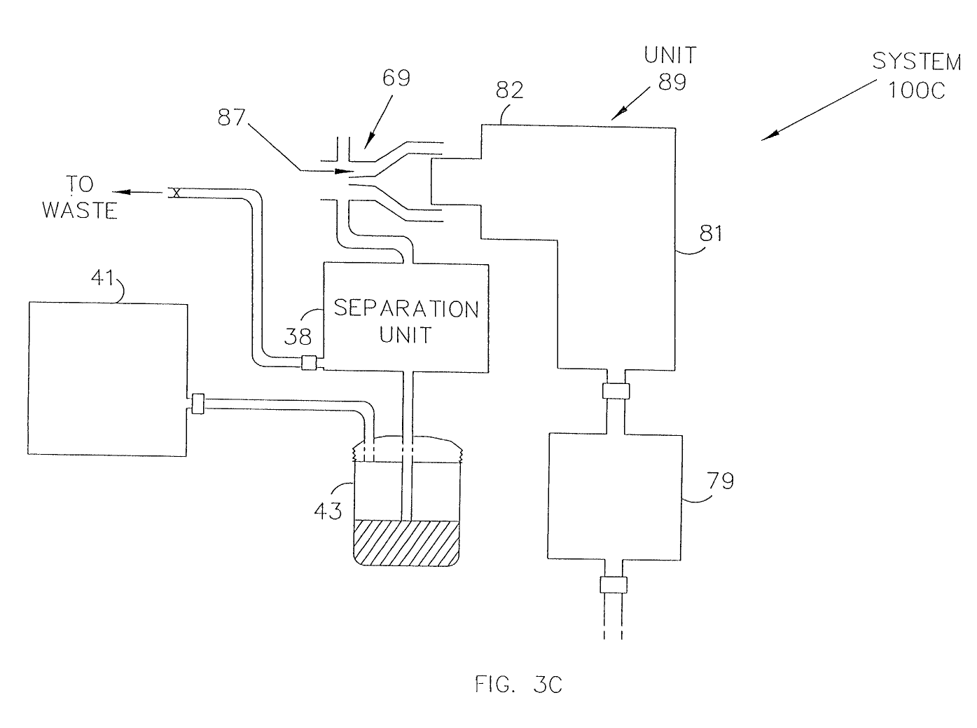

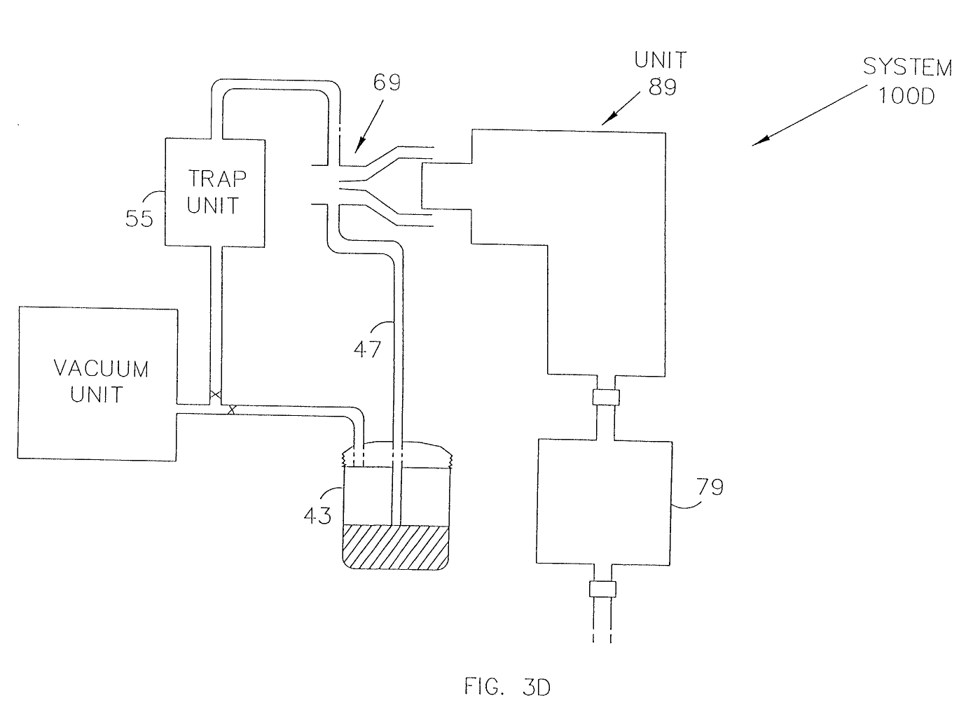

FIG. 3A shows a schematic representation of a fluid jet blood sampling system 100A′ according to another embodiment of the invention. In this embodiment, a pressure supply unit 79 receives fluid from a fluid supply unit. The pressure supply unit 79 pressurizes the fluid and is connected to a fluid jet unit 89. The pressure supply unit 79 and the fluid jet unit 89 make up a fluid jet device 99, which is capable of providing a high pressure fluid jet. The pressure supply unit 79 and the fluid jet unit 89 may each be controlled by one or more valves (not shown).

The fluid jet unit 89 includes a handpiece 85 and a jet nozzle 87. The handpiece 85 includes a housing 84, a switch (or trigger) 83, a barrel portion 82 and a stock portion 81. During use of the fluid jet blood sampling system, the jet nozzle 87 is sealably coupled to the barrel portion 81.

Also, during use of the fluid jet blood sampling system, a cowling or hood 69 may be sealably attached to the barrel portion 82 of the fluid jet unit 89. During a blood sampling procedure, the cowling 69 is brought into close proximity or physical contact with the skin of the individual at a locus from which a blood sample is to be drawn. The cowling 69 is constructed in such a way and to appropriate dimensions such that the entire skin surface at the locus is surrounded by the cowling 69. In this manner, distribution of fluid from the fluid jet, as well as contamination from blood in the form of droplets or an aerosol, fragments of skin tissue, debris, and the like is minimized or prevented.

When a fluid jet from the fluid jet unit 89 perforates the skin and/or breaks at least one blood vessel within or beneath the skin, the first quantity of blood that accumulates on the skin is likely to be mixed with fluid from the fluid jet, skin cells, etc. Depending on factors such as the nature of the blood sample to be collected and the type of analysis/analyses to be performed on the blood sample, a sample of the first quantity of blood may be collected via a collection unit. Alternatively, in situations where a whole blood sample is required, or where the presence of fluid from the fluid jet must be avoided, the first quantity of blood may be removed using the drying unit 59. The term “whole blood” means a sample of blood which is not fractionated, diluted or otherwise contaminated or mixed with non-blood components. After a suitable period of time, a second quantity of blood may be allowed to accumulate at the locus, and a sample of the second quantity of blood may be collected. The second quantity of blood should be substantially free of contaminants.

In the embodiment shown in FIG. 3A, the cowling 69 includes at least one collection coupling piece 68 for coupling a collection duct 47 thereto. The collection duct 47 is further coupled to a collection reservoir (or vessel) 43 for containing a blood sample obtained from a patient. The collection reservoir 43 is further coupled to a collection vacuum unit 41 for providing suction power to transfer a quantity of blood from within the cowling 69 to the collection reservoir 43. The transfer of blood from within the cowling 69 to the collection reservoir 43 may be controlled by one or more valves (not shown). The transfer of blood within components of the collection unit may also be controlled by one or more additional valves (also not shown).

In the embodiment shown in FIG. 3B, the cowling 69 may include one or more drying coupling pieces for coupling a drying duct 57 to the cowling 69. The drying duct 57 is coupled to a trap unit 55 for trapping unwanted liquid from within the cowling 69. The trap unit 55 is coupled to a drying vacuum unit 53 for providing suction power to transfer unwanted liquid or material from within the cowling 69 to the trap unit 55. The unwanted liquid material may comprise, for example, a mixture of fluid emitted as the fluid jet, a first quantity of blood, suspended skin tissue fragments, and the like. The trap unit 55 may serve to contain or hold the unwanted liquid material until such time as the unwanted liquid material is to be disposed of or discarded. The transfer of fluid emitted as the fluid jet, blood, fragments of skin tissue, and the like, from within the cowling 69 to the trap unit 55 may be controlled by one or more valves (not shown).

FIG. 3C shows a fluid jet blood sampling system 100C according to another embodiment of the invention. In this embodiment, the fluid jet blood sampling system includes a separation unit 38 for separating unwanted fluid and materials from a blood sample or from a cellular fraction of blood. The use of the separation unit eliminates the need for a drying unit. The separation unit 38 may be coupled to a collection duct 47, and be located between the collection reservoir 43 and the cowling 69. The separated fluid from the fluid jet may be disposed of subsequent to separation of fluid from a blood sample. The blood sample would then be collected in a collection reservoir 43.

FIG. 3D shows a fluid jet blood sampling system according to another embodiment of the invention. In this embodiment, the fluid jet blood sampling system uses a single vacuum unit for both removing unwanted liquid composed from within the cowling 69, and for collection of a blood sample in a collection reservoir 43. Valves could be used to first apply suction through the trap unit 55 to remove unwanted liquid and skin fragments from the collection locus on the skin. The valves could then be reconfigured to apply suction through the collection vessel 43 to collect a blood sample from the skin.

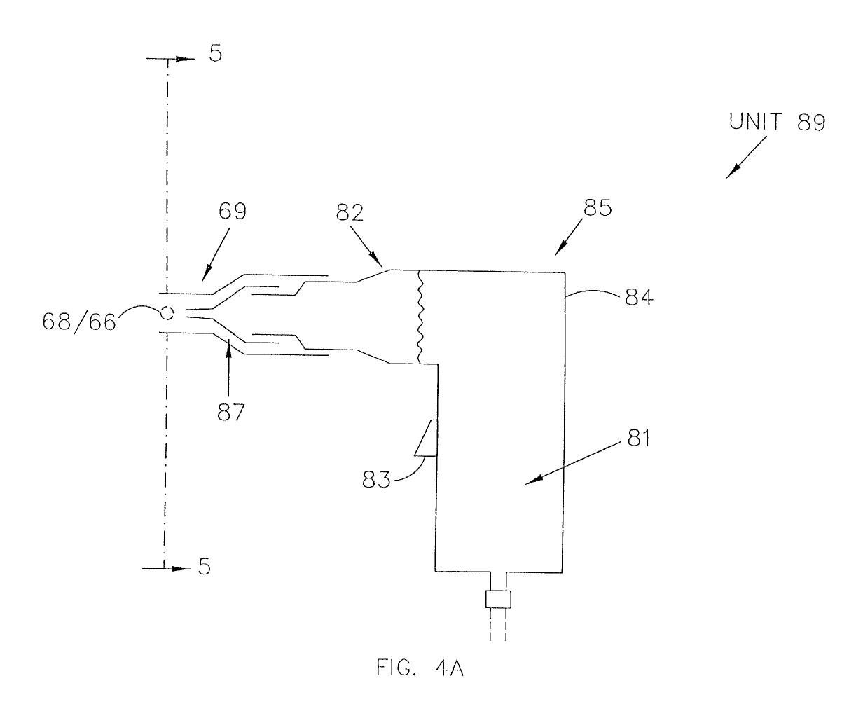

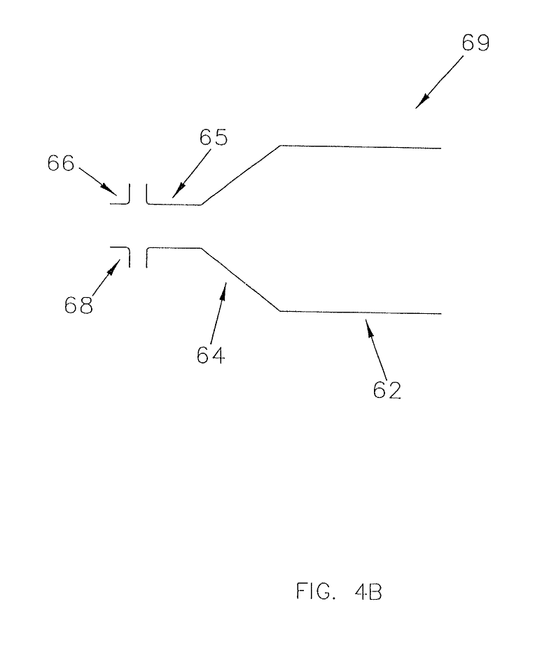

FIG. 4A is a more detailed representation of fluid jet unit 89, showing a handpiece 85, including a housing 84 together with an attached jet nozzle 87 and a cowling 69. A switch 83 controls the flow of the fluid jet from the jet nozzle 87 and is arranged on the stock portion 81 of the handpiece 85.

A fluid jet blood sampling device or system according to the invention may include a disposable handpiece or may include a handpiece which includes both disposable components and non-disposable components. Preferably the disposable components are kept to a minimum in order to minimize costs of consumable/disposable components on a per unit basis. However, in preferred embodiments of the invention, the jet nozzle 87 is disposable. In the preferred embodiments, after the fluid jet blood sampling system has been used to collect a sample of blood from a patient, the jet nozzle 87 is discarded and disposed of (as biohazardous waste). To this end, the jet nozzle 87 is configured so that it is readily detachable from the barrel portion 82. A new (clean and sterile) jet nozzle 87 is provided for the next patient. A disposable jet nozzle 87 may be provided at equal or less cost per unit compared with lancets and other sharp instruments used in conventional blood sampling.

In contrast to conventional blood sampling techniques, which employ sharps to penetrate the skin, a jet nozzle 87 is constructed so as to be non-sharp, and the jet nozzle will not penetrate human skin at even relatively high impact forces. For this reason, the risk to medical and other personnel of accidentally cutting themselves during handling and disposal of used jet nozzles 87 is minimized. Preferably the jet nozzles 87 are constructed from plastic material, such as molded polypropylene, polyethylene, polyurethane, polyvinylchloride, etc.

In some embodiments of the invention, the jet nozzle 87 may be provided with a one way valve (not shown) in order to preclude the possibility of components upstream from jet nozzle 87 (e.g., the barrel portion 82) from becoming contaminated with blood from a particular patient.

In some embodiments of the invention, the entire barrel portion 82 of the handpiece 85 may be configured so that it is disposable. In this instance, the jet nozzle and the barrel portion would be used on only one patient on a single occasion, and then the entire assembly would be discarded. In this way the risks of cross contamination of patients, or their respective blood samples, with blood from other individuals is minimized.

In other embodiments of the invention, the jet nozzle 87 may include an adjustable feature which allows the user to selectively vary characteristics of the fluid jet emanating therefrom. For instance, the pressure supply unit of a device embodying the invention may supply fluid at a substantially constant pressure and volume. The jet nozzle, however, may be adjustable to allow the user to vary the pressure at which the fluid jet is emitted, and/or the pattern of the fluid jet as it leaves the jet nozzle. This adjustment could also affect the duration of the fluid jet.

In still other embodiments of the invention, a plurality of different jet nozzles having different characteristics may be available for use with the same fluid supply device. For instance, one type of jet nozzle might output a first type of fluid jet at a first pressure, whereas a second type of jet nozzle, attached to the same fluid supply unit, could output a fluid jet at a different pressure. This could be done by varying the diameter and/or shape of the aperture in the jet nozzle, or by other means.

In preferred embodiments of the invention, the pressure supply unit 79 provides fluid at a pressure of from about 8,000 to about 11,000 psi. The jet nozzle 87 has a diameter in the range of from about 50-100 μm, and preferably a diameter of about 80 μm.

FIG. 4B is a more detailed representation of one embodiment of a cowling or hood 69, showing a conical portion 64 linking a proximal end 62 and a distal end 65. The distal end 65 includes at least one collection coupling piece 68. The cowling 69 may be constructed of suitable materials and in such a way so as to be disposable and quickly and easily attached/detached from fluid jet unit 89.

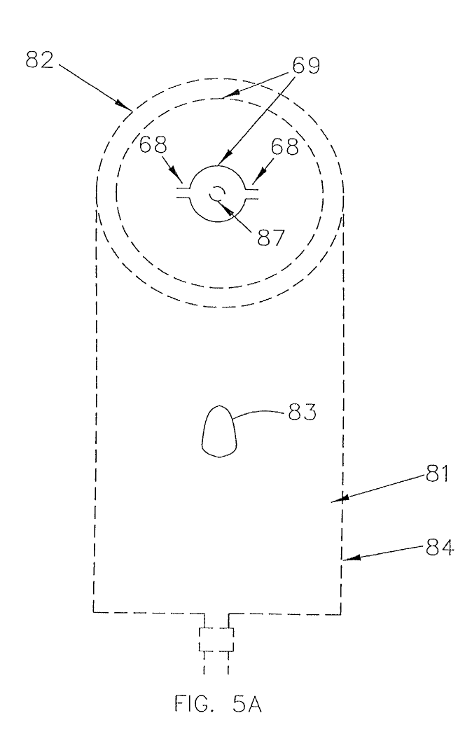

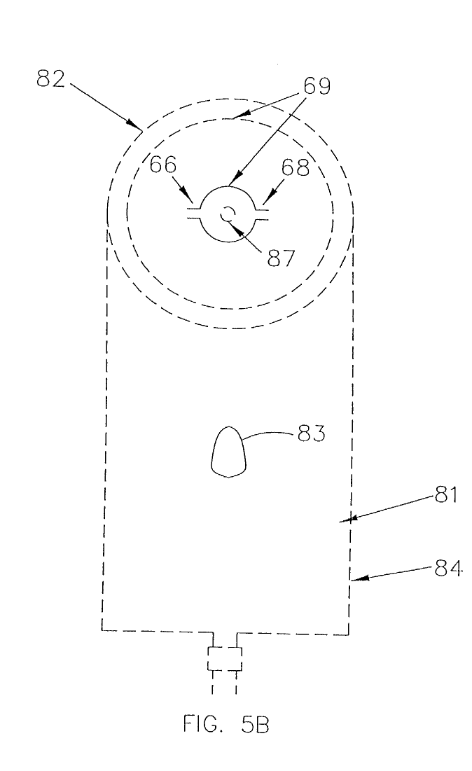

FIG. 5A shows a frontal view of a fluid jet unit (such as the fluid jet unit of FIG. 4A) showing the jet nozzle 87 and the cowling 69 attached to a barrel portion 82 of a handpiece 85. In the embodiment of FIG. 5A, the cowling 69 bears a pair of symmetrically spaced collection coupling pieces 68, each of which is configured to be coupled to collection ducts. The cowling 69 may have a single collection coupling piece 68, or several collection coupling pieces 68. Also, the collection coupling pieces 68 may be spaced regularly (equidistant from each other) or irregularly around the circumference of the distal end 65 of the cowling 69.

FIG. 5B shows a frontal view of a fluid jet unit (such as the fluid jet unit of FIG. 4A) having a jet nozzle 87 and a cowling 69 attached to a barrel portion 82 of a handpiece 85. This embodiment is similar to the one shown in FIG. 5A, but this embodiment has a cowling 69 with a single collection coupling piece 68, and the addition of a drying coupling piece 66. The cowling 69 may include a single drying coupling piece 66, or several drying coupling pieces 66. Also, the drying coupling pieces 66 may be spaced regularly (equidistant from each other) or irregularly around the circumference of the distal end 65 of the cowling 69.

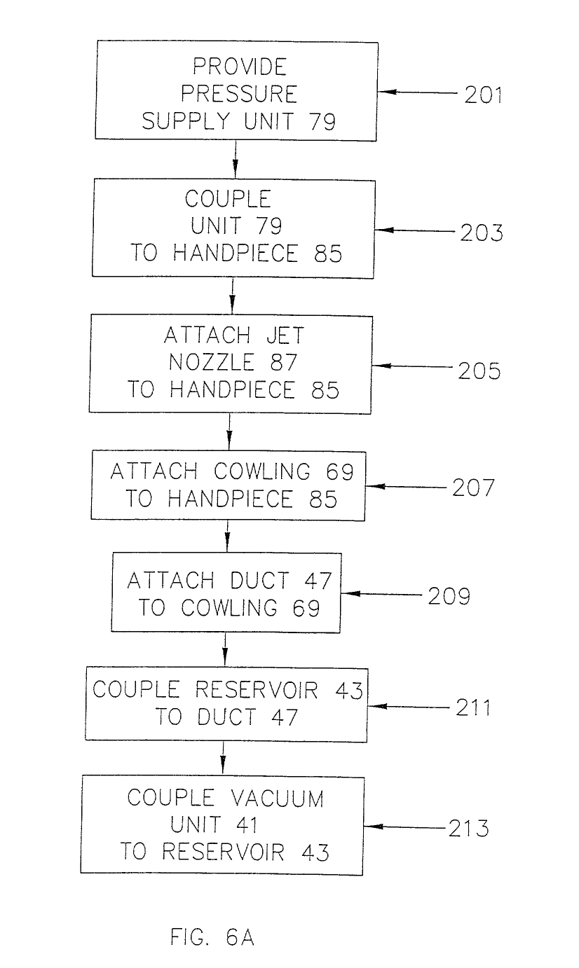

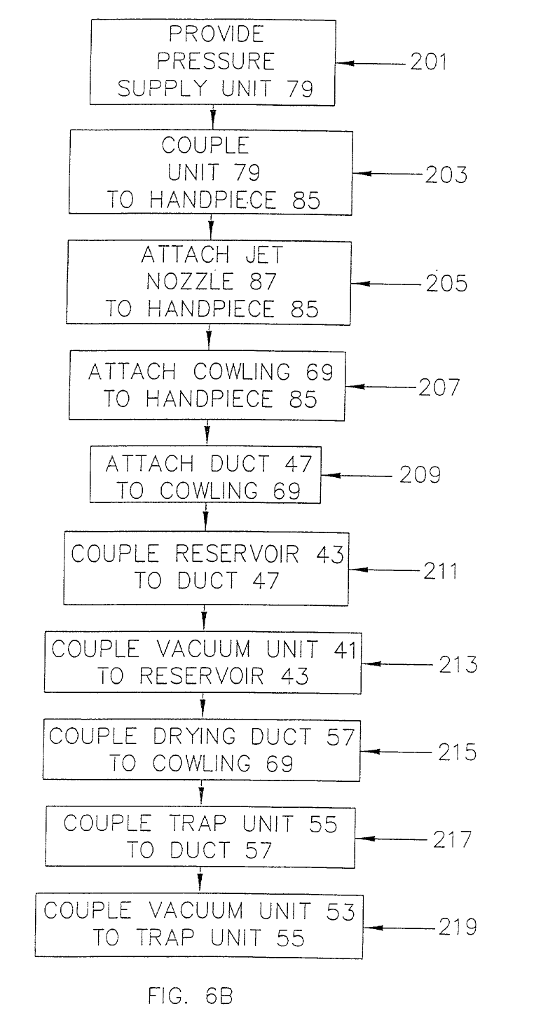

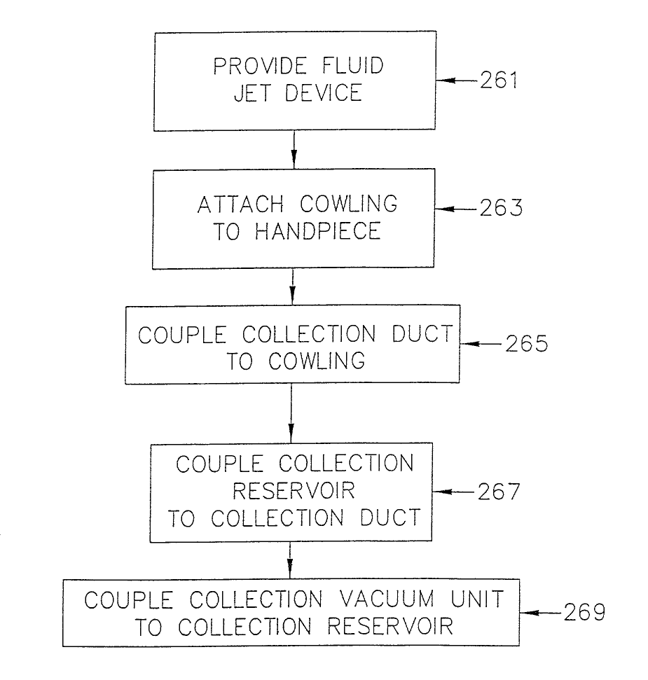

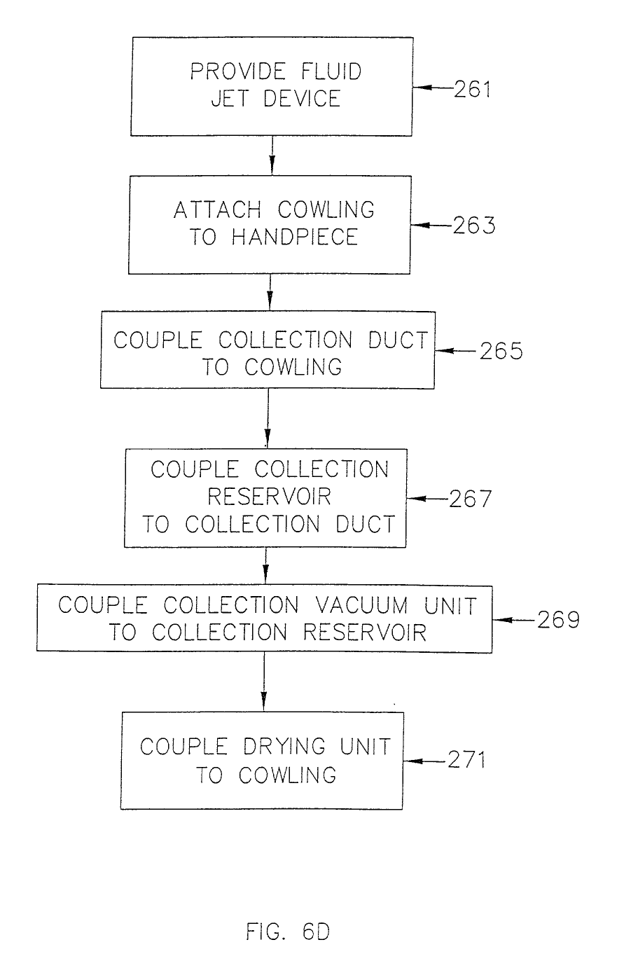

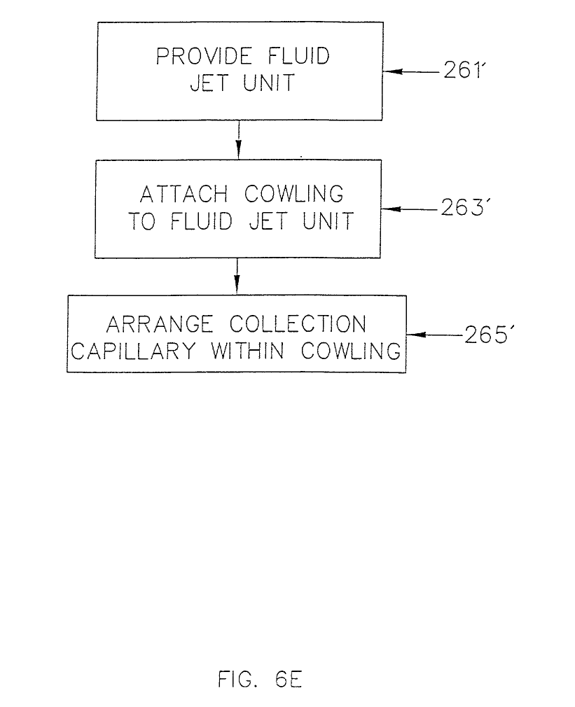

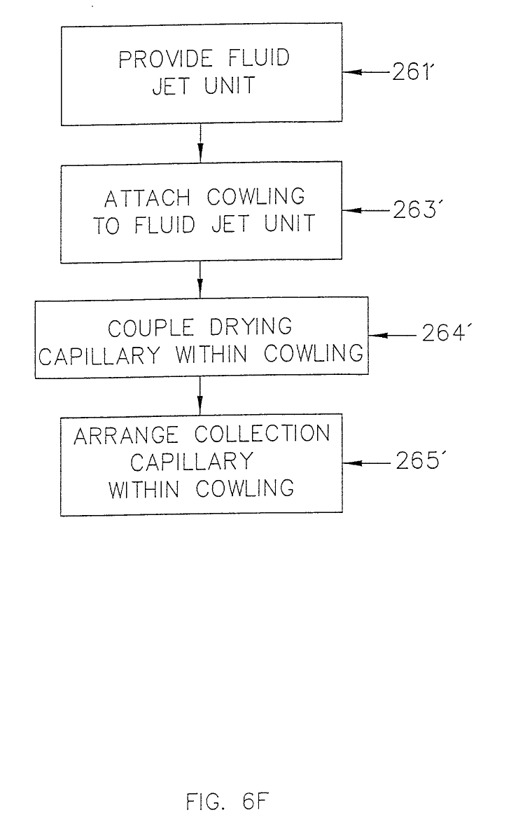

FIG. 6A summarizes the steps involved in a method for making a fluid jet blood sampling system according to one embodiment of the invention. Step 201 involves providing a pressure supply unit 79. Step 203 involves coupling the pressure supply unit 79 to a handpiece 85. In step 205, the jet nozzle 87 is attached to the handpiece 85. Step 207 entails attaching a cowling 69 to the handpiece 85. A collection duct 47 is attached to the cowling 69 in step 209. Step 211 involves coupling a collection reservoir 43 to the collection duct 47. Thereafter, a collection vacuum unit 41 is coupled to the collection reservoir 43 in step 213.

FIG. 6B summarizes the steps involved in making a fluid jet blood sampling system according to another embodiment of the invention, in which steps 201 through 213 are common to the method depicted in FIG. 6A. The embodiment of FIG. 6B further includes the steps of, after step 213, coupling a drying duct 57 to the cowling 69 in step 215. A trap unit 55 is coupled to the drying duct 57 in step 217. A drying vacuum unit 53 is coupled to the trap unit 55 in step 219.

FIG. 6C summarizes the steps involved in assembling a fluid jet blood sampling system according to another embodiment of the invention. Step 261 involves providing a fluid jet device 99. In step 263, a cowling 69 is attached to a handpiece 85 of the fluid jet device 99. A collection duct 47 is coupled to the cowling 69 in step 265. A collection reservoir 43 is coupled to the collection duct 47 in step 267. A collection vacuum unit 41 is coupled to the collection reservoir 43 in step 269.

FIG. 6D summarizes the steps involved in assembling a fluid jet blood sampling system according to another embodiment of the invention. In this method, steps 261 through 269 are the same as in the method depicted in FIG. 6C. The method depicted in FIG. 6D, however, also includes the step 271, which involves coupling a drying unit 59 to the cowling 69.

FIG. 6E summarizes the steps involved in assembling a fluid jet blood sampling system according to another embodiment of the invention. In this method, step 261′ involves providing a fluid jet unit that includes a disposable jet nozzle and a handpiece. Step 263′ involves attaching a cowling to the handpiece of the fluid jet unit. Step 265′ involves arranging a collection capillary within the cowling.

FIG. 6F summarizes the steps involved in assembling a fluid jet blood sampling system according to another embodiment of the invention. In this method, step 261′ involves providing a fluid jet unit that includes a disposable jet nozzle and a handpiece. Step 263′ involves attaching a cowling to the handpiece of the fluid jet unit. Step 264′ involves coupling a drying capillary within the cowling. Step 265′ involves arranging a collection capillary within the cowling.

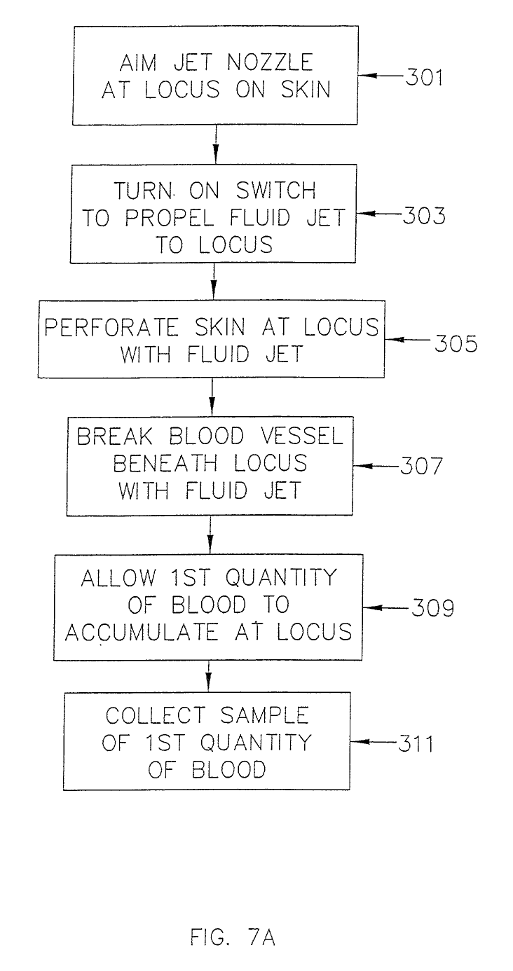

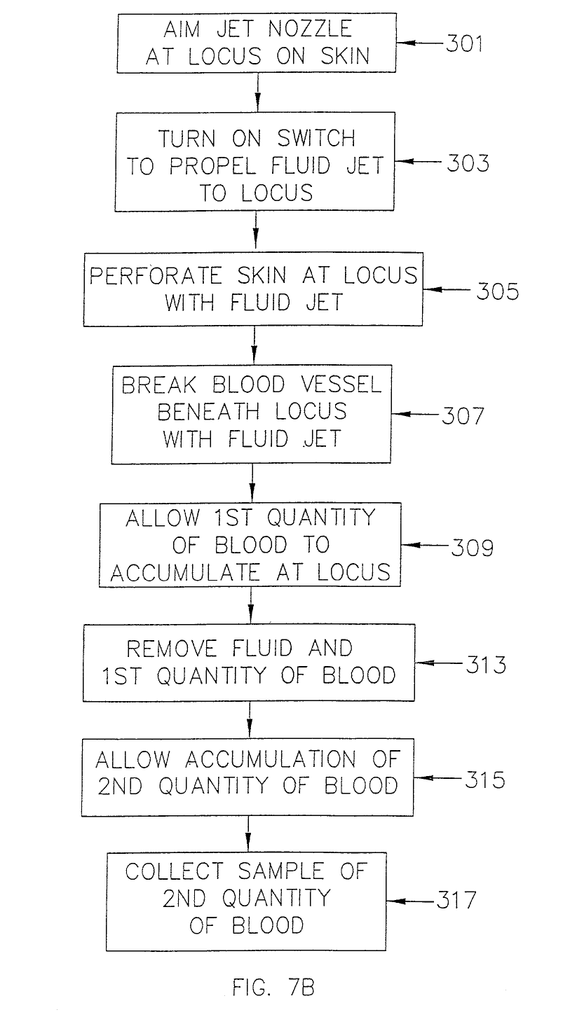

FIG. 7A summarizes the steps involved in a method for obtaining a blood sample from a patient according to another embodiment of the invention. In step 301, a jet nozzle of a fluid jet unit is aimed at a locus on the skin of a patient. In step 303, a switch on the fluid jet unit is turned on to cause one or more short duration fluid jets to be propelled at the locus on the skin of the patient. In step 305, the skin of the patient is perforated at the locus by the fluid jet. Step 307 involves breaking at least one blood vessel beneath the locus on the skin of the patient by means of the fluid jet. Steps 303, 305 and 307 will all happen substantially simultaneously when a switch of the device is triggered by the user. Step 309 involves allowing the accumulation of a first quantity of blood at the locus. Thereafter, a sample of the first quantity of blood may be collected from the locus on the skin in step 311.

FIG. 7B shows another embodiment of the invention. In this embodiment, steps 301 through 309 are basically the same as in the method depicted in FIG. 7A. In this method, however, in step 313, unwanted liquid such as fluid from the fluid jet and the first quantity of blood are removed from the locus on the skin of the patient. In step 315, a second quantity of blood is allowed to accumulate at the locus. Thereafter, in step 317, a sample of the second quantity of blood is collected.

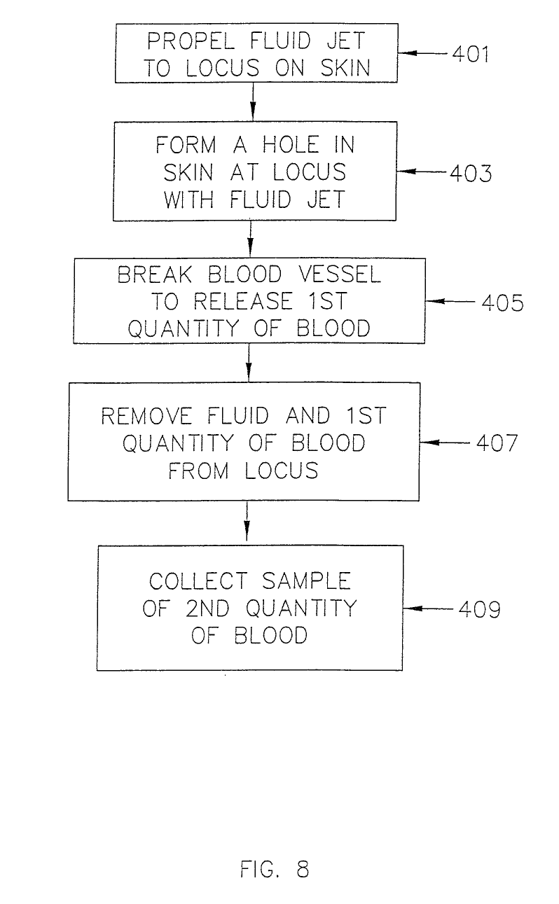

FIG. 8 summarizes the steps involved in a method for obtaining a blood sample from a patient, using a fluid jet blood sampling system, according to yet another embodiment of the invention. Step 401 involves propelling a fluid jet to a locus on the skin of a patient from which a blood sample is to be withdrawn. Step 403 involves forming a hole in the skin at the locus by means of the fluid jet. In step 405, at least one blood vessel is broken by the fluid jet to release a first quantity of blood from the at least one broken blood vessel at the locus. In step 407, excess or unwanted liquid, such as fluid from the fluid jet and the first quantity of blood, is removed from the locus on the skin of the patient. After the accumulation of a second quantity of blood at the locus, a sample of the second quantity of blood is collected in step 409.

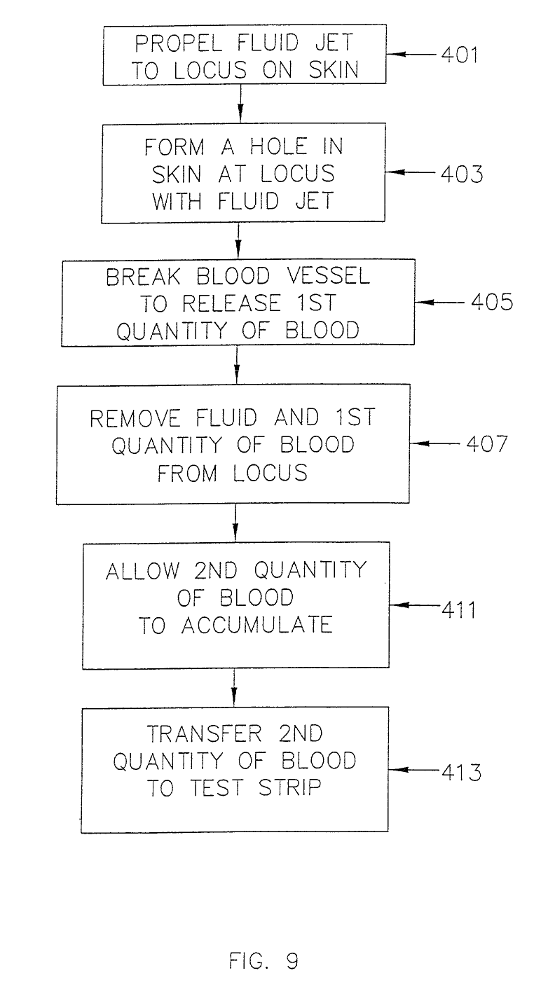

FIG. 9 summarizes the steps involved in a method for obtaining a blood sample from a patient, using a fluid jet blood sampling system, according to yet another embodiment of the invention. In this method, steps 401 through 407 are essentially the same as in the method depicted in FIG. 8. In this method, after step 407, step 411 involves allowing at least one drop of a second quantity of blood to accumulate on the surface of the skin. Thereafter, step 413 involves collecting or transferring a sample of the second quantity of blood to a test strip.

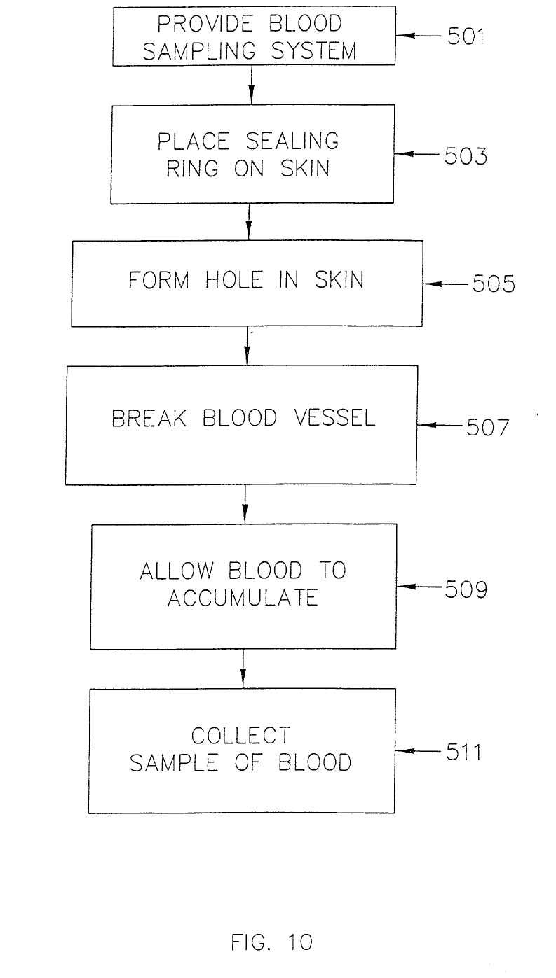

FIG. 10 summarizes the steps involved in a method for obtaining a blood sample from a patient, using a fluid jet blood sampling system, according to yet another embodiment of the invention. Step 501 involves providing a fluid jet blood sampling system that includes a control unit and an integrated jet nozzle/cowling. The integrated jet nozzle/cowling provided in step 501 includes a sealing ring and a jet nozzle, wherein the jet nozzle is for providing a fluid jet capable of forming a hole in skin. Step 503 involves placing the sealing ring on the skin surface of a patient. Step 505 involves forming a hole in the skin by means of the fluid jet provided by the jet nozzle. Step 507 involves breaking at least one blood vessel adjacent to the hole in the skin, again by means of the fluid jet. Step 509 involves allowing at least one drop of blood to accumulate on the skin surface. Step 511 involves collecting a sample of the at least one drop of blood allowed to accumulate in step 509.

Alternate methods embodying the invention, may include the steps 501 through 511 as described above with reference to FIG. 10, and may include the additional step of, prior to the step 511, removing the sealing ring from the skin surface. Step 511 may include collecting a sample of the at least one drop of blood with a secondary device. A secondary device for collecting a sample of the at least one drop of blood according to step 511 may include a relatively narrow bore tube, or a test strip, or the like.

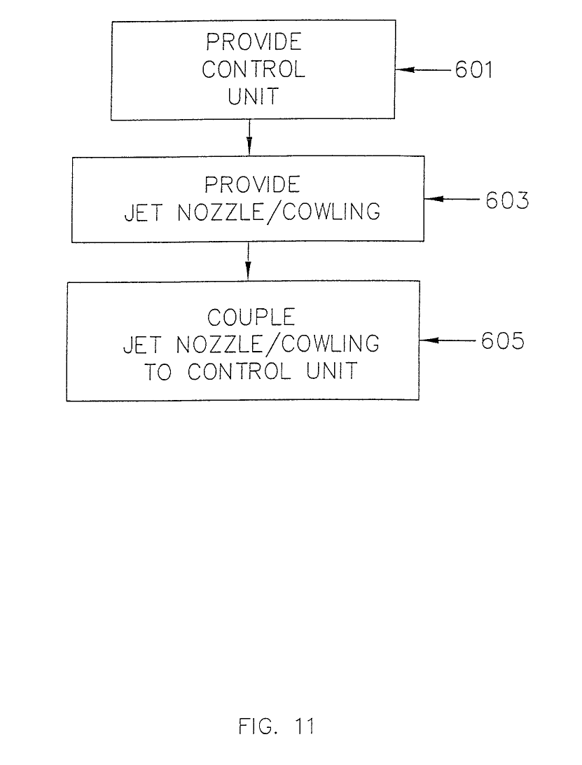

FIG. 11 summarizes the steps involved in a method for assembling a fluid jet blood sampling system, according to another embodiment of the invention. Step 601 involves providing a control unit capable of providing a pressurized fluid to a jet nozzle. Step 603 involves providing an integrated jet nozzle/cowling which includes a channel unit, a sealing ring, and a jet nozzle having a jet nozzle distal end. Step 605 involves functionally coupling the control unit to the integrated jet nozzle/cowling.

In alternate methods embodying the invention, step 601 may involve providing a control unit which is capable of providing pressurized fluid to a jet nozzle at a pressure ranging from about 1,000 psi to about 15,000 psi. According to another embodiment of a method for assembling a fluid jet blood sampling system, step 603 may involve providing an integrated jet nozzle/cowling which is constructed as a single piece of molded plastic.

While the fluid jet blood sampling system and methods for obtaining blood samples have been described herein primarily with respect to humans, it is to be understood that the various embodiments of the instant invention are also applicable to non-human animals, for example, in veterinary medicine and/or biomedical research applications.

The foregoing embodiments are merely exemplary and are not to be construed as limiting the present invention. The methods of the present invention can be readily applied to other types of apparatuses. The description of the present invention is intended to be illustrative, and not to limit the scope of the claims. Many alternatives, modifications, and variations will be apparent to those skilled in the art.

What is claimed is:

1. A fluid jet blood sampling system, comprising:
a control unit for supplying a pressurized fluid;
a jet nozzle functionally coupled to said control unit, wherein the jet nozzle and the control unit are configured to eject the pressurized fluid as at least one short duration fluid jet that is capable of perforating a patient's skin; and
a sealing unit for containing at least one of the fluid from the fluid jet and any initial outflow of blood escaping the patient's skin.

2. The fluid jet blood sampling system of claim 1, wherein the jet nozzle and sealing unit form an integrated jet nozzle/cowling unit.

3. The fluid jet blood sampling system of claim 2, wherein said integrated jet nozzle/cowling is disposable and is molded as a single piece of plastic.

4. The fluid jet blood sampling system of claim 2, wherein the integrated jet nozzle/cowling further comprises at least one channel unit for collecting at least one of the fluid from the fluid jet and an initial outflow of blood escaping the patient's skin.

5. The fluid jet blood sampling system of claim 4, wherein the channel unit comprises at least one capillary tube arranged between the jet nozzle and the sealing unit.

6. The fluid jet blood sampling system of claim 4, wherein said integrated jet nozzle/cowling further includes a crown, said crown disposed circularly between said jet nozzle and said channel unit, said crown including one or more crown furrows for directing fluid toward said channel unit.

7. The fluid jet blood sampling system of claim 1, wherein said jet nozzle has a diameter ranging from about 50 to about 100 μm.

8. The fluid jet blood sampling system of claim 1, wherein the jet nozzle and the control unit are configured to supply the at least one fluid jet at a pressure ranging from about 1,000 to about 15,000 psi.

9. The fluid jet blood sampling system of claim 1, wherein the jet nozzle is configured to direct the at least one fluid jet toward the patient's skin at an acute angle relative to a skin surface.

10. The fluid jet blood sampling system of claim 9, wherein the angle is adjustable.

11. The fluid jet blood sampling system of claim 1, wherein a duration of the at least one fluid jet is adjustable.

12. The fluid jet blood sampling system of claim 1, wherein the system is configured such that a user can adjust the pressure at which the at least one fluid jet is provided.

13. The fluid jet blood sampling system of claim 1, wherein the system is configured to provide a plurality of fluid jets during a single blood sampling operation, and wherein the control unit is configured so that a user can adjust the number of fluid jets that are provided during a single blood sampling operation.

14. The fluid jet blood sampling system of claim 1, wherein the control unit is configured to allow a user to adjust the volume of fluid in each at least one fluid jet.

15. The fluid jet blood sampling system of claim 1, wherein the jet nozzle includes a backflow control device that prevents fluid from passing from an output end of the jet nozzle to the control unit.

16. The fluid jet blood sampling system of claim 15, wherein the backflow control device comprises a one way check valve.

17. The fluid jet blood sampling system of claim 1, wherein the jet nozzle is configured so that a user can adjust the pressure at which a fluid jet is provided.

18. The fluid jet blood sampling system of claim 17, wherein the jet nozzle is configured so that a diameter of the jet nozzle can be changed by a user.

19. The fluid jet blood sampling system of claim 1, wherein the fluid includes at least one of an anti-bacterial agent, an anti-infective agent, and an anesthetic.

20. A blood sampling system, comprising:
a jet nozzle configured to emit a fluid as at leas, one short duration fluid jet, said fluid jet being capable of perforating the skin of a patient such that blood can flow from the skin; and
a cowling surrounding the jet nozzle.

21. The system of claim 20, wherein the jet nozzle and the cowling are formed from a single piece of molded plastic.

22. The system of claim 20, further comprising a collection unit for collecting a sample of blood that flows from the skin of the patient.

23. The system of claim 22, wherein the collection unit comprises a collection duct and a collection reservoir, the collection duct being coupled to said cowling and transferring blood from the cowling to the collection reservoir.

24. The system of claim 20, further comprising a pressure supply unit functionally connected to said jet nozzle for providing pressurized fluid to the jet nozzle.

25. The system of claim 24, wherein the system is configured so that a user can adjust the pressure at which the at least one fluid jet is emitted.

26. The system of claim 24, wherein the pressure supply unit is configured so that a user can adjust the volume of fluid in each at least one fluid jet.

27. The system of claim 24, wherein the system is configured to emit a plurality of fluid jets during a single blood sampling operation, and wherein the pressure supply unit is configured so that a user can adjust the number fluid jets emitted by the jet nozzle during a single blood sampling operation.

28. The system of claim 20, wherein the jet nozzle is configured so that a user can adjust the pressure at which the at least one fluid jet is emitted.

29. The system of claim 20, further comprising a drying unit for removing unwanted materials from the patient's skin.

30. The system of claim 29, wherein the drying unit comprises:
a trap unit for trapping the unwanted materials; and
a drying unit for removing the unwanted materials from the trap unit.

31. The system of claim 20, wherein the fluid jet is configured to emit the at least one fluid jet at a pressure between approximately 1000 psi and approximately 15,000 psi.

32. A method for obtaining a blood sample from a patient, comprising the steps of:
forming a hole in the patient's skin with at least one short duration fluid jet;
allowing blood to accumulate on a surface of the patient's skin; and
collecting a sample of the blood.

33. The method of claim 32, wherein said step of collecting a sample of the blood comprises collecting a sample of the blood with at least one of a test strip and a collection vessel.

34. The method of claim 32, wherein the step of forming a hole in the patient's skin with at least one short duration fluid jet also breaks a blood vessel beneath a surface of the skin.

35. The method of claim 32, wherein the step of forming a hole in the patient's skin comprises forming the hole with a plurality of short duration fluid jets.

36. The method of claim 32, wherein the step of forming a hole in the patient's skin comprises forming the hole with at least one short duration fluid jet that is emitted at a pressure of between approximately 1000 psi and approximately 15,000 psi.

37. The method of claim 32, wherein the step of forming a hole in the patient's skin comprises emitting the at least one short duration fluid jet with a device having a jet nozzle and a cowling that surrounds the jet nozzle, wherein the jet nozzle and the cowling are formed from a single piece of molded plastic.

38. The method of claim 32, further comprising the steps of:
allowing a first quantity of blood to collect on the patient's skin after the hole is formed in the patient's skin with the at least one fluid jet;
removing the first quantity of blood, and any fluid from the at least one fluid jet from the patient's skin; and
allowing a second quantity of blood to accumulate on the patient's skin, wherein the step of collecting a sample comprises collecting a sample of the second quantity of blood.
